(19) United States
(12) Patent Application Publication
(10) Pub. No.: US 2017/0354396 A1
(43) Pub. Date: Dec. 14, 2017

(54) WIRELESS ULTRASOUND PROBE ADAPTER

(71) Applicant: General Electric Company, Schenectady, NY (US)

(72) Inventor: Warren Lee, Niskayuna, NY (US)

(21) Appl. No.: 15/177,349

(22) Filed: Jun. 9, 2016

(51) Int. Cl. A61B 8/00; A61B 8/08; A61B 8/14

(52) U.S. Cl. CPC A61B 8/4472; A61B 8/4477; A61B 8/14; A61B 8/5207; A61B 8/461; A61B 8/4438; A61B 8/565; A61B 8/4411; A61B 8/546

(57) ABSTRACT

An ultrasound wireless probe adapter is presented. The adapter includes a first coupling unit configured to detachably couple the adapter to ultrasound probe assemblies, a second coupling unit configured to wirelessly couple the adapter to a smart device, and a microcontroller. The microcontroller is configured to wirelessly communicate with the smart device to accept user inputs, generate and transmit one of excitation signals and control and configuration signals to the ultrasound probe assemblies based on the user inputs and a category of the ultrasound probe assemblies to initiate emission of acoustic signals towards a region of interest in a subject, receive echo signals generated by the ultrasound probe assemblies in response to one of the transmitted excitation signals or the transmitted control and configuration signals, and process received beam signals based on a processing capability of the smart device to generate one of partially-processed image data and fully-processed image data.

### BACKGROUND

Embodiments of the present specification generally relate to an ultrasound system, and more specifically to a wireless ultrasound probe adapter configured for use with different types of ultrasound probes.

Various noninvasive diagnostic imaging modalities are capable of producing cross-sectional images of organs or vessels inside the body. An imaging modality that is well suited for such noninvasive imaging is ultrasound. Ultrasound diagnostic imaging systems are in widespread use by cardiologists, obstetricians, radiologists and others for examining the heart, a developing fetus, internal abdominal organs, and other anatomical structures. These systems operate by transmitting waves of ultrasound energy into the body. The transmitted waves impinge on tissue interfaces resulting in reflection of ultrasound echoes from the tissue interfaces. The reflected ultrasound echoes are then translated into structural representations of portions of the body through which the ultrasound waves are directed.

In conventional ultrasound imaging, objects of interest such as internal tissues and blood are scanned using planar ultrasound beams or slices. A linear array transducer, also known as a one-dimensional array, is conventionally used to scan a thin slice by narrowly focusing the transmitted and received ultrasound in an elevated direction and steering the transmitted and received ultrasound throughout a range of angles in an azimuth direction. A transducer having a linear array of transducer elements can operate in this manner to provide a two-dimensional image representing a cross-section through a plane that is perpendicular to a face of the transducer.

Linear arrays can also be used to generate three-dimensional images (for example, “volumetric” images), by rotating or translating the one-dimensional array of transducer elements in the elevation direction or by sweeping the array through a range of angles extending in the elevation direction. Volumetric ultrasound images can also be conventionally obtained by using a two-dimensional array transducer to steer the transmitted and received ultrasound about two axes.

A conventional ultrasound probe assembly typically includes a system connector, cabling, and a transducer. These conventional ultrasound probes are designed and manufactured for use in specific applications. For example, scanning of different parts of the body calls for use of different types of ultrasound probes. Use of different probes for different applications increases the amount of cabling and electronic circuitry that need to be duplicated in each probe, thereby leading to higher costs for the manufacturer and end user. In addition, the huge volume of cables and the need for carrying multiple bulky probe assemblies restrict the portability of compact systems such as laptop-based ultrasound systems. Furthermore, even though the currently available ultrasound systems are becoming increasingly miniaturized such that the system electronics are integrated inside the probe handle, utilization of the existing conventional probes in a compact, low cost, easily upgradable ultrasound system is a challenging task.

### BRIEF DESCRIPTION

In accordance with aspects of the present specification, an ultrasound wireless probe adapter is presented. The ultrasound probe adapter includes a first coupling unit configured to detachably couple the probe adapter to one or more ultrasound probe assemblies, a second coupling unit configured to wirelessly couple the probe adapter to a smart device, and a microcontroller operatively coupled to the first coupling unit and the second coupling unit. The microcontroller is configured to wirelessly communicate with the smart device to accept user inputs, generate and transmit one of excitation signals and control and configuration signals to the one or more ultrasound probe assemblies based on the user inputs and a category of the one or more ultrasound probe assemblies to initiate emission of acoustic signals towards a region of interest in a subject, receive echo signals generated by the one or more ultrasound probe assemblies in response to one of the transmitted excitation signals or the transmitted control and configuration signals, and process received beam signals based on a processing capability of the smart device to generate one of partially-processed image data and fully-processed image data, where the received beam signals are generated based on the received echo signals. Furthermore, the probe adapter is configured to wirelessly transmit one of the partially-processed image data and the fully-processed image data to the smart device for generation and display of an image of the region of interest in the subject.

In accordance with another aspect of the present specification, an ultrasound imaging system is presented. The ultrasound imaging system includes one or more ultrasound probe assemblies, a smart device and an ultrasound wireless probe adapter. The ultrasound wireless probe adapter includes a first coupling unit configured to detachably couple the probe adapter to the one or more ultrasound probe assemblies, a second coupling unit configured to wirelessly couple the probe adapter to the smart device, and a microcontroller operatively coupled to the first coupling unit and the second coupling unit. The microcontroller is configured to wirelessly communicate with the smart device to accept user inputs, generate and transmit one of excitation signals and control and configuration signals to the one or more ultrasound probe assemblies based on the user inputs and a category of the one or more ultrasound probe assemblies to initiate emission of acoustic signals towards a region of interest in a subject, receive echo signals generated by the one or more ultrasound probe assemblies in response to the transmitted excitation signals or the transmitted control and configuration signals, and process received beam signals based on a processing capability of the smart device to generate one of partially-processed image data and fully-processed image data, where the received beam signals are generated based on the received echo signals. Furthermore, the probe adapter is configured to wirelessly transmit one of the partially-processed image data and the fully-processed image data to the smart device for generation and display of an image of the region of interest in the subject.

In accordance with yet another aspect of the present specification, a method for imaging is presented. The method includes coupling an ultrasound wireless probe adapter to a cable connector of one or more ultrasound probe assemblies, wherein the probe adapter comprises a first coupling unit configured to detachably couple the probe adapter to the one or more ultrasound probe assemblies, a second coupling unit configured to wirelessly couple the probe adapter to a smart device, and a microcontroller operatively coupled to the first coupling unit and the second coupling unit. The microcontroller is configured to wirelessly communicate with the smart device to accept user inputs, generate and transmit one of excitation signals and control and configuration signals to the one or more ultrasound probe assemblies based on the user inputs and a category of the one or more ultrasound probe assemblies to initiate emission of acoustic signals towards a region of interest in a subject, receive echo signals generated by the one or more ultrasound probe assemblies in response to the transmitted excitation signals or the transmitted control and configuration signals, process received beam signals based on a processing capability of the smart device to generate one of partially-processed image data and fully-processed image data, where the received beam signals are generated based on the received echo signals, wirelessly coupling the probe adapter to the smart device via the second coupling unit, authorizing a user of the probe adapter, generating an image based on one of the partially-processed image data and the fully-processed image data, and displaying the image on the smart device.

### DRAWINGS

These and other features and aspects of embodiments of the present invention will become better understood when the following detailed description is read with reference to the accompanying drawings in which like characters represent like parts throughout the drawings, wherein:

FIG. 1 is a block diagram of a system for imaging a region of interest in a subject using an exemplary wireless probe adapter, in accordance with aspects of the present specification;

FIG. 2 is a block diagram of one embodiment of the wireless probe adapter for use in the system of FIG. 1, in accordance with aspects of the present specification;

FIG. 3 is a block diagram of a smart device for use in the imaging system of FIG. 1; and

FIG. 4 is a flowchart of a method for imaging a region of interest in a subject using the system of FIG. 1 having the wireless ultrasound probe adapter of FIG. 2, in accordance with aspects of the present specification.

### DETAILED DESCRIPTION

Unless defined otherwise, technical and scientific terms used herein have the same meaning as is commonly understood by one of ordinary skill in the art to which this disclosure belongs. The terms “a” and “an” do not denote a limitation of quantity, but rather denote the presence of at least one of the referenced items. The term “or” is meant to be inclusive and mean one, some, or all of the listed items. The use of “including,” “comprising” or “having” and variations thereof herein are meant to encompass the items listed thereafter and equivalents thereof as well as additional items. The terms “control system” or “controller” may include either a single component or a plurality of components, which are either active and/or passive and are connected or otherwise coupled together to provide the described function or functions.

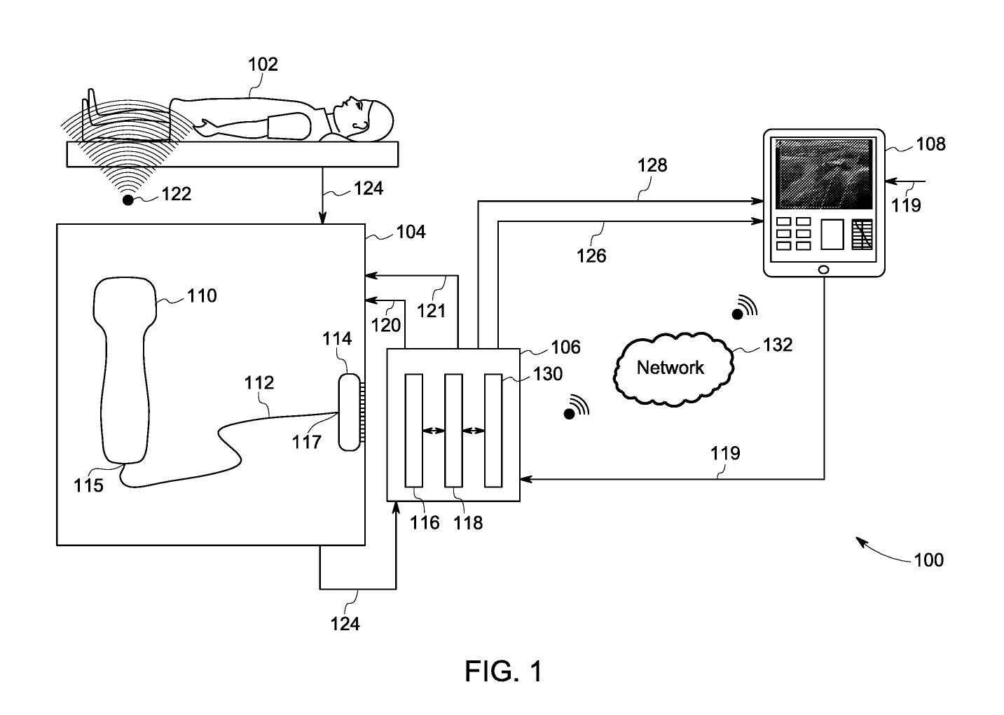

FIG. 1 is a diagrammatic illustration of a system 100 for imaging a region of interest (ROI) in a subject 102, in accordance with aspects of the present specification. The subject 102, for example, may be a patient or an object. In a presently contemplated configuration, the system 100 is an ultrasound imaging system. The system 100 includes an ultrasound probe assembly 104, an exemplary ultrasound wireless probe adapter 106, and a smart device 108. The ultrasound probe assembly 104 may be operatively coupled to the probe adapter 106. In addition, the probe adapter 106 may be wirelessly coupled to the smart device 108. It may be noted that the terms ultrasound wireless probe adapter, wireless probe adapter, and probe adapter may be used interchangeably.

The ultrasound probe assembly 104 includes a probe 110, a cable 112, and a cable connector 114. By way of a non-limiting example, the probe 110 may include a linear array ultrasound probe, a phased array ultrasound probe, a convex array ultrasound probe, and the like. The probe 110 is coupled to the cable connector 114 via the cable 112. For example, a first end 115 of the cable 112 is coupled to the probe 110, and a second end 117 of the cable 112 is coupled to the cable connector 114.

As previously noted, the system 100 also includes the probe adapter 106. The probe adapter 106 is characterized by a portable and compact size. In certain embodiments, a size of the probe adapter 106 is equal to a size of the cable connector 114. It may be noted that for ease of illustration, in FIG. 1, the size of the probe adapter 106 is depicted as being larger than the size of the cable connector 114.

The probe adapter 106 is configured to be detachably couplable to one or more ultrasound probe assemblies. In the presently contemplated configuration, the probe adapter 106 is shown as being operatively coupled to the ultrasound probe assembly 104. The ultrasound probe assembly 104 may be a conventional wired probe assembly. As used herein, the phrase “wired ultrasound probe assembly” refers to an ultrasound probe assembly that entails use of a wired connection with an ultrasound console or a smart device for functioning of the ultrasound probe assembly. The exemplary probe adapter 106 is configured to convert conventional wired probe assemblies to wireless probe assemblies. Accordingly, operatively coupling the probe adapter 106 to the wired ultrasound probe assembly 104 converts the wired ultrasound probe assembly 104 to a wireless ultrasound probe assembly 104. As used herein, the phrase “wireless ultrasound probe assembly” refers to an ultrasound probe assembly that does not entail use of a wired connection to an ultrasound console or a smart device for functioning. Consequent to the use of the probe adapter 106, the ultrasound probe assembly 104 is wirelessly coupled to the ultrasound console or the smart device 108.

In accordance with further aspects of the present specification, the functioning of the probe adapter 106 may be configurable based on a category of ultrasound probe 110 being used. In particular, the functioning of the probe adapter 106 may be adapted based on processing capabilities of the ultrasound probe 110 being used. As will be appreciated, in certain scenarios, sophisticated ultrasound probes may include active transmit/receive (TX/RX) electronics in the probe handle. However, some conventional ultrasound probes may not include active TX/RX electronics in the probe handle. Accordingly, in one exemplary embodiment, the probe adapter 106 includes hardware and/or software that are essential for ultrasound imaging. In particular, the probe adapter 106 may include circuitry for enabling transmission, reception, and/or processing of ultrasound signals. In certain embodiments, the probe adapter 106 may be configured to perform the functions that are traditionally performed by the TX/RX electronics.

As will be appreciated, there exist different categories of ultrasound probe assemblies. The ultrasound probe assembly 104 may be categorized based on a function, size, shape, application, presence or absence of TX/RX electronics and/or technology of the ultrasound probe assembly 104. By way of a non-limiting example, the different categories of ultrasound probe assemblies may include a linear array ultrasound probe assembly, a phased array ultrasound probe assembly, a convex array ultrasound probe assembly, and the like. Another category of the ultrasound probe assembly 104 may be differentiated based on a presence or absence of the TX/RX electronics in the ultrasound probe assembly 104. In accordance with aspects of the present specification, the probe adapter 106 may be configured to be used with these different categories of ultrasound probe assemblies to enable these ultrasound probe assemblies to operate as wireless ultrasound probe assemblies.

Additionally, in accordance with further aspects of the present specification, a desired amount of processing by the probe adapter 106 of signals received from the ultrasound probe assembly 104 may be determined based on a processing capability of the smart device 108 being used. By way of example, use of a smart device 108 having a higher performance/processing capability may allow a faster processing of the signals/data by the smart device 108, thereby resulting in higher frame rates. In this scenario, the choice of whether to perform the image processing via use of the wireless probe adapter 106 or the smart device 108 is dependent on the relative performance of the smart device 108 and the probe adapter 106. Accordingly, in certain embodiments, the probe adapter 106 may be configured to compare the performance of image processors in each of the probe adapter 106 and the smart device 108.

More particularly, the probe adapter 106 may further be configured to reorganize/split the tasks to be performed to optimize the performance of the system 100. For example, based on the comparison if it is determined that the processing capability of the probe adapter 106 is better than that of the smart device 108, then the received signals may be fully processed by the probe adapter 106 to generate fully-processed image data representative of an image. Furthermore, the fully-processed image data is communicated to the smart device 108 for display. However, based on the comparison, if it is determined that the processing capability of the probe adapter 106 is lower than that of the smart device 108, then the received signals may be partially processed by the probe adapter 106 and partially-processed image data may be communicated to the smart device 108. In this scenario, the smart device 108 may be configured to further process the partially-processed image data to generate an image for display.

In accordance with further aspects of the present specification, the probe adapter 106 may include hardware and/or software that are essential for ultrasound imaging. In particular, the probe adapter 106 may include circuitry for enabling transmission, reception, and/or processing of ultrasound signals. In one embodiment, the probe adapter 106 includes a first coupling unit 116, a second coupling unit 130, and a microcontroller unit 118 or a microcontroller 118. The first coupling unit 116 is configured to detachably couple the probe adapter 106 to the ultrasound probe assembly 104. Additionally, the first coupling unit 116 may also be configured to facilitate coupling the probe adapter 106 to the different categories of ultrasound probe assemblies. The first coupling unit 116, for example, may be an electrical connector, such as a male connector, a female connector, and the like.

Moreover, in some embodiments, the cable connector 114 of the ultrasound probe assembly 104 may be selected based on a type of the first coupling unit 116. Alternatively, in some other embodiments, the first coupling unit 116 may be selected to enable coupling the probe adapter 106 to a given cable connector 114. By way of example, if the first coupling unit 116 is a male type of connector, then a female type of connector may be used as the cable connector 114. In a similar fashion, a female type of connector may be employed as the first coupling unit 116 if the cable connector 114 is a male type of connector.

As noted hereinabove, the probe adapter 106 includes the microcontroller unit or microcontroller 118. The microcontroller 118 is operatively coupled to the first coupling unit 116. Also, the microcontroller 118 wirelessly communicates with the smart device 108 via a wireless network 132 established by the second coupling unit 130 of the probe adapter 106. In one example, the microcontroller 118 is configured to wirelessly communicate with the smart device 108 to accept user inputs 119. It may be noted that the user inputs 119 may be used to control operation of the probe adapter 106 and the probe assembly 104. Additionally, in some embodiments, the microcontroller 118 may be an integrated chip, a chip scale package, and the like.

During a transmit operation, the microcontroller 118 is configured to perform at least one of transmitting data/information and/or controls and generating and transmitting control and configuration signals or excitation signals to array elements of the probe 110 for transmit beam formation. In a similar manner, during a receive operation, the microcontroller 118 is configured to perform at least one of filtering, amplifying, compensating for attenuation, digitizing an echo voltage stream, receiving data and/or information from the ultrasound probe assembly 104, and forming the receive beam.

As noted hereinabove, the operation of the probe adapter 106 may be adapted based on the category/type of ultrasound probe assembly 104. In particular, the microcontroller 204 may be configured to generate excitation signals 120 or control and configuration signals 121 based on at least one of a configuration or processing capability of the ultrasound probe assembly 104, the user inputs 119, and the category of the ultrasound probe assembly 104. More specifically, the microcontroller 118 is configured to adapt the operation of the probe adapter 106 based on the category of the ultrasound probe assembly, the configuration or processing capability of the ultrasound probe assembly, and a type of imaging requested by a user of the smart device 108. For example, if the ultrasound probe assembly 104 does not include active TX/RX electronics, the microcontroller 118 is configured to generate the excitation signals 120. Additionally, the microcontroller 118 is configured to transmit these excitation signals 120 directly to transducer array elements in the probe 110. Moreover, the excitation signals 120 merely excite the transducer array elements of the ultrasound probe assembly 104 resulting in generation of acoustic signals 122. Accordingly, in this example, the probe adapter 106 is capable of performing the functions of the active TX/RX electronics.

In other embodiments, if the ultrasound probe assembly 104 includes active TX/RX electronics, then the TX/RX electronics in the probe adapter 106 is bypassed, and the microcontroller 118 is configured to generate and transmit control and configuration signals 121 to the ultrasound probe assembly 104. By way of a non-limiting example, the control and configuration signals 121 may include information related to a frequency, pulse repetition frequency, coding of the acoustic signals 122, an amplitude of the acoustic signals 122, a duration of the acoustic signals 122, timing of the excitation of the transducer array elements of the probe assembly 104, or combinations thereof.

In response to the receipt of the excitation signals 120 or the control and configuration signals 121 from the microcontroller 118, the ultrasound probe assembly 104 emits the acoustic signals 122 towards the ROI in the subject 102. Once the acoustic signals 122 impinge on the ROI, at least a portion of the acoustic signals 122 are reflected by the ROI resulting in generation of echo signals 124. The echo signals 124 are received by the ultrasound probe assembly 104. Furthermore, the ultrasound probe assembly 104 may transmit the echo signals 124 to the microcontroller 118. Accordingly, the microcontroller 118 receives the echo signals 124 generated in response to the transmitted control and configuration signals 121 or the excitation signals 120 from the ultrasound probe assembly 104.

In accordance with aspects of the present specification, the probe adapter 106 is configured to generate received beam signals (not shown) based on the received echo signals 124. As noted hereinabove, the probe adapter 106 and the microcontroller 118 in particular may be configured to determine the desired amount/nature of processing of the beam signals received from the ultrasound probe assembly 104 based on the processing capability of the smart device 108. In one embodiment, if the processing capability of the microcontroller 118 is better than that of the smart device 108, the microcontroller 118 is configured to process the received beam signals to generate fully-processed image data 126. The fully-processed image data 126 is representative of an image of the ROI in the subject 102. However, if the processing capability of the smart device 108 is better than that of the probe adapter 106, the microcontroller 118 may only partially process the received beam signals to generate partially-processed image data 128. The partially-processed image data 128 may be subsequently processed by the smart device 108 to generate the image of the ROI in the subject 102. It may be noted that use of the partially-processed image data 128 for generating an image of the ROI in the subject 102 may entail further processing prior to use in the generation of the image of the ROI in the subject 102.

As previously noted, the probe adapter 106 also includes the second coupling unit 130. The second coupling unit 130 is operatively coupled to the microcontroller 118. By way of a non-limiting example, the second coupling unit 130 may be a wireless adapter. The second coupling unit 130 is configured to wirelessly couple the probe adapter 106 to the smart device 108. The wireless coupling of the probe adapter 106 to the smart device 108 enables the probe adapter 106 to wirelessly communicate with the smart device 108. The wireless communication between the probe adapter 106 and the smart device 108 may include transmission of the partially-processed image data 128 or the fully-processed image data 126.

Moreover, as previously noted, the system 100 further includes the smart device 108. The smart device 108, for example, may be a processing device, a smart mobile phone, a laptop, a personal computer, a tablet, a personal digital assistant, and the like. The smart device 108 may serve as a user interface to allow a clinician/user to enter the user inputs 119. In addition, the smart device 108 may also provide ability to display an image and/or image data.

The probe adapter 106 is configured to wirelessly couple the otherwise wired ultrasound probe assembly 104 to the smart device 108. In one example, the probe adapter 106 may be configured to wirelessly couple the ultrasound probe assembly 104 to the smart device 108 via the wireless network 132. Also, in one embodiment, the smart device 108 may be configured to transmit inputs and controls to the probe adapter 106 via the wireless network 132. Additionally, the smart device 108 may be configured to transfer inputs, data, information, and/or controls over the wireless network 132 via the probe adapter 106 to the ultrasound probe assembly 104. Furthermore, the smart device 108 may receive information and data over the wireless network 132 from the ultrasound probe assembly 104.

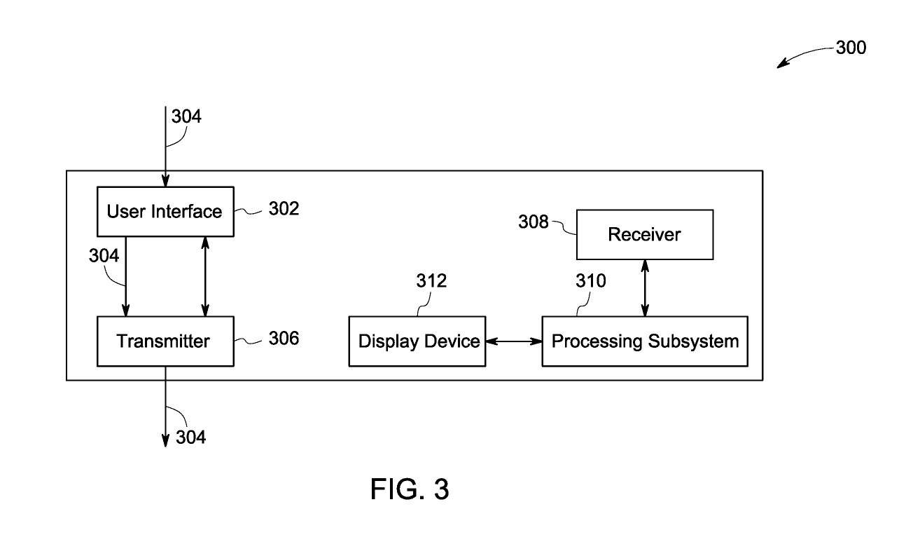

In certain embodiments, the smart device 108 may be configured to receive the partially-processed image data 128 from the probe adapter 106. In this example, the smart device 108 may be configured to process the partially-processed image data 128 to generate an image of the ROI in the subject 102. In another embodiment, the smart device 108 may be configured to receive the fully-processed image data 126 from the probe adapter 106. In this example, the smart device 108 may be configured to display the image based on the fully-processed image data received from the probe adapter 106. The smart device 108 will be described in greater detail with reference to FIG. 3.

Implementing the wireless probe adapter 106 that may be coupled to the cable connector 114 of a conventional ultrasound probe assembly 104 as described hereinabove allows for wireless operation of the ultrasound probe assembly 104 in conjunction with the smart device 108. The probe adapter 106 may provide a cost-effective solution to upgrade a huge installed base of existing conventional probes to a wireless (untethered), compact, low cost, and easily upgradable ultrasound imaging system.

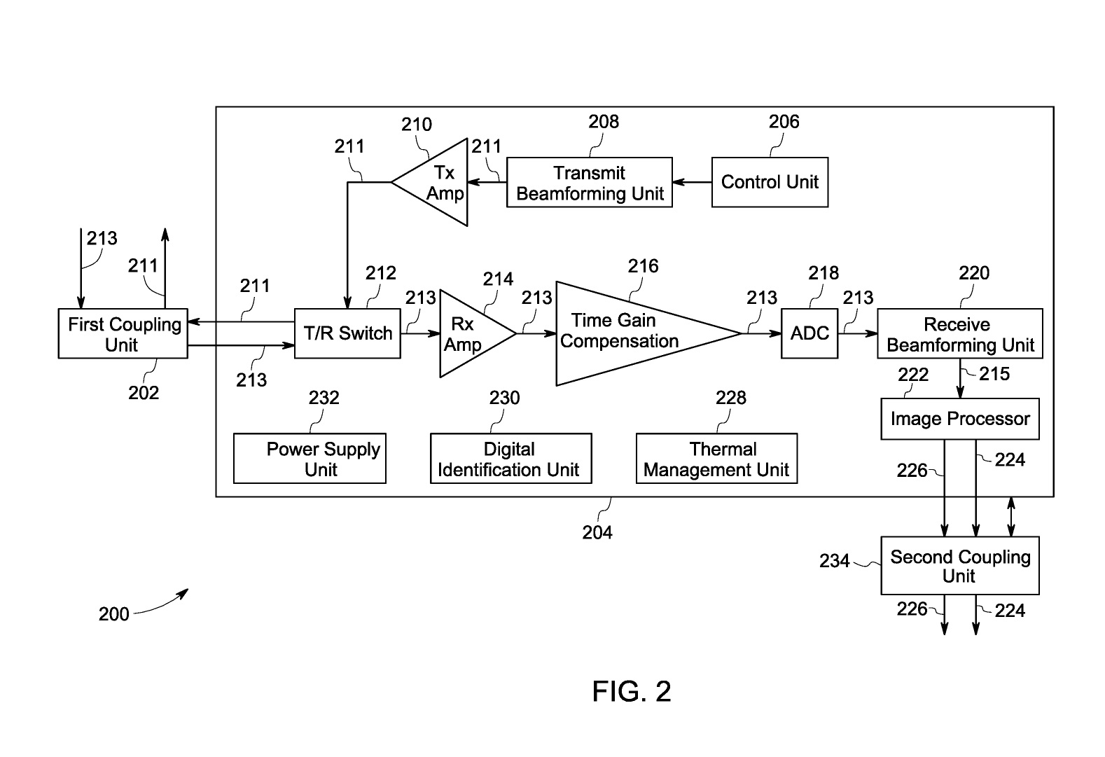

Referring now to FIG. 2, a block diagram of one embodiment of a probe adapter 200 for use in the system 100 of FIG. 1, in accordance with aspects of the present specification, is presented. The probe adapter 200, for example may be the probe adapter 106 of FIG. 1. As previously noted with reference to FIG. 1, the probe adapter 106 includes the first coupling unit 116, the microcontroller 118, and the second coupling unit 130. In the example of FIG. 2, the wireless probe adapter 200 is shown as including a first coupling unit 202, a microcontroller unit or microcontroller 204, and a second coupling unit 234. In one embodiment, the first coupling unit 202, the microcontroller 204, and the second coupling unit 234 may be respectively representative of the first coupling unit 116, the microcontroller 118, and the second coupling unit 130 of FIG. 1. Although for ease of illustration FIG. 2 depicts various components of the probe adapter 200, it may be noted that the probe adapter 200 and the microcontroller 204 may have additional or fewer components, and the flow of information and signals between the components may vary in comparison to the flow of information and signals described with reference to FIG. 2.

The first coupling unit 202 may be configured to detachably couple the probe adapter 200 to an ultrasound probe assembly (not shown), such as the ultrasound probe assembly 104 of FIG. 1. Also, the first coupling unit 202 may be configured to couple the probe adapter 200 to different types/categories of ultrasound probe assemblies.

As previously noted, the probe adapter 200 additionally includes the microcontroller 204. It may be noted that a single component of the microcontroller 204 may perform functions of multiple components, and hence this single component may be used to replace the multiple components of the probe adapter 200. In a presently contemplated configuration, the microcontroller 204 includes a control unit 206, a transmit beamforming unit 208, a transmit amplifier 210, a transmit/receive switch 212, a receive amplifier 214, a time gain compensation amplifier 216, an analog to digital (ADC) converter 218, a receive beamforming unit 220, and an image processor 222. Each of the control unit 206 and the image processor 222 may include an integrated chip, at least one arithmetic logic unit, and/or a microprocessor configured to perform computations, and/or retrieve data stored in memory. It may be noted that although the microcontroller 204 is depicted as having the transmit beamforming unit 208 and the receive beamforming unit 220, in certain embodiments, the function of both the transmit and receive beamforming units 208, 220 may be performed by a single beamforming unit.

In the presently contemplated configuration, the control unit 206 is operatively coupled to the transmit beamforming unit 208. The control unit 206 wirelessly communicates with the smart device 108 in order to accept user inputs 119. Based on the user inputs 119, the control unit 206 may generate and transmit command data to the transmit beamforming unit 208. The transmit command data in turn may be used for generating excitation signals 211. Moreover, the excitation signals 211 are employed to generate acoustic signals of a desired shape and direction.

The transmit beamforming unit 208 receives commands from the control unit 206 and generates the excitation signals 211. The excitation signals 211 are used to excite the transducer array elements of the ultrasound probe assembly in order to generate the acoustic signals of the desired shape and direction. The transmit beamforming unit 208 may be operatively coupled to the transmit amplifier 210. The transmit amplifier 210 amplifies the excitation signals 211 to generate signals of a desired voltage. Additionally, the transmit amplifier 210 transmits the amplified excitation signals 211 via the transmit/receive switch 212 and the first coupling unit 202 to an ultrasound probe assembly (not shown) coupled to the probe adapter 200.

For ease of explanation, in the example of FIG. 2, it is assumed that the ultrasound probe assembly coupled to the probe adapter 200 does not include active TX/RX electronics and hence the probe adapter 200 is configured to generate and transmit the excitation signals 211. Accordingly, the probe adapter 200 is configured to perform the functions that are otherwise performed by the active TX/RX electronics in the ultrasound probe assembly. However, if the ultrasound probe assembly coupled to the probe adapter 200 includes active TX/RX electronics, then the probe adapter 200 may not be required to perform the operations typically performed by the active TX/RX electronics. Additionally, in this example, the probe adapter 200 is configured to generate and transmit control and configuration signals to the ultrasound probe assembly.

The transmission of the excitation signals 211 or the control and configuration signals results in transmission of acoustic signals (not shown) towards a subject/patient (not shown). The acoustic signals are backscattered off tissue and blood samples within the patient to generate echo signals 213. The echo signals 213 are received by the microcontroller 204 from the ultrasound probe assembly. Particularly, the echo signals 213 are received by the receive amplifier 214 via the first coupling unit 202 and the transmit/receive switch 212 in the microcontroller 204. The receive amplifier 214 amplifies the echo signals 213. As shown in FIG. 2, the receive amplifier 214 is operatively coupled to the time gain compensation amplifier 216. The time gain compensation amplifier 216 amplifies the echo signals 213 to compensate for attenuation in the patient's tissue. Further, the time gain compensation amplifier 216 is operatively coupled to the ADC converter 218 that digitizes the echo signals 213. The digitized echo signals 213 are thereafter transmitted to the receive beamforming unit 220.

The digitized echo signals 213 are received by the receive beamforming unit 220. The receive beamforming unit 220 uses command data received from the control unit 206 to form a received beam at a desired steering angle. In particular, the receive beamforming unit 220 operates on the digitized echo signals 213 via use of filtering, directing, focusing, and/or apodizing in accordance with the instructions of the command data from the control unit 206 to generate received beam signals 215. The received beam signals 215 are representative of the received beam corresponding to sample volumes along a scan line within the patient. Information such as phase, amplitude, and timing information of the received echo signals 213 from various transducer elements in the ultrasound probe assembly are used to generate the received beam signals 215.

The receive beamforming unit 220 is in turn operatively coupled to the image processor 222. The image processor 222 may receive the received beam signals 215 from the receive beamforming unit 220. In certain embodiments, the image processor 222 may be operatively coupled to a smart device (not shown), such as the smart device 108 of FIG. 1. The image processor 222 may be configured to process the received beam signals 215. Particularly, the image processor 222 may be configured to fully or partially process the received beam signals 215 based on a processing capability of the smart device. In one embodiment, at least one of the probe adapter 200, the image processor 222, and the smart device may be configured to determine a desired amount of processing of the received beam signals 215 by the image processor 222 based on a comparison of the processing capabilities of the probe adapter 200 and the smart device. By way of example, at least one of the probe adapter 200, the image processor 222, and the smart device may select between the partial processing and full processing of the received beam signals 215 by the image processor 222 based on a comparison of a processing capability of the image processor 222 with the processing capability of the smart device.

In one embodiment, the image processor 222 is configured to partially process the received beam signals 215 to generate partially-processed image data 224 based on the processing capability of the smart device. For example, if the processing capability of the smart device is substantially faster than a processing capability of the probe adapter 200 or the image processor 222, then the image processor 222 may partially process the received beam signals 215 to generate the partially-processed image data 224. The partially-processed image data 224 may not be representative of an image, and hence may necessitate further processing prior to use in the generation of an image of an ROI in the patient. The smart device may subsequently process the partially-processed image data 224 to generate an image (not shown) for display.

Alternatively, based again on the processing capability of the smart device the image processor 222 may also be configured to fully process the received beam signals 215 to generate fully-processed image data 226. The fully-processed image data 226 is representative of the image of the ROI in the patient. For example, if the processing capability of the smart device is worse than the processing capability of the image processor 222 or the smart device is incapable of processing the received beam signals 215, then the image processor 222 may fully process the received beam signals 215 to generate the fully-processed image data 226. Fully processing the received beam signals 215 may include scan conversion to reformat the received beam signals 215 into image form, pre-processing (for example, spatial compounding and 3D processing), storing image frames, post-processing into gray or color scales, and the like. The fully-processed image data 226 is representative of an image of the ROI in the patient.

In accordance with further aspects of the present specification, the microcontroller 204 also includes a digital identification unit 230 configured to authorize a user of the probe adapter 200. By way of example, the digital identification unit 230 may require the user of the probe adapter 200 to provide an input such as a unique password and/or biometric data to authenticate the user prior to allowing usage of the probe adapter 200.

In certain embodiments, the microcontroller 204 may also include a thermal management unit 228 configured to manage a temperature of the probe adapter 200. Additionally, the microcontroller 204 may include a power supply unit 232 configured to supply electric power to the probe adapter 200. In one embodiment, the power supply unit 232 may be a battery. In one example, the power supply unit 232 may be a rechargeable battery.

Furthermore, the probe adapter 200 includes the second coupling unit 234 operatively coupled to the microcontroller 204. The second coupling unit 234 is configured to wirelessly couple the probe adapter 200 to the smart device. The second coupling unit 234, for example, may be a wireless adapter. The wireless coupling of the probe adapter 200 to the smart device enables wireless transmission of portions of the partially-processed image data 224 and/or the fully-processed image data 226 from the probe adapter 200 to the smart device for generation and/or display of an image of the ROI in the patient.

Turning now to FIG. 3, a block diagram of a smart device 300 for use in the imaging system 100 of FIG. 1 is presented. The smart device 300, for example, may be a processing device, a smart mobile phone, a laptop, a personal digital assistant, and the like. The smart device 300, for example may be the smart device 108 of FIG. 1. In one embodiment, the smart device 300 includes a user interface 302 configured to enable a user to enter user inputs and/or controls 304. The user inputs, for example may be the user inputs 119. These inputs and/or controls 304 may be communicated to a probe adapter that is wirelessly coupled to the smart device 300. The inputs and/or controls 304, for example, may include details regarding a ROI in a subject to be scanned, details of the subject, preference(s) of the user, controls required for initiation and execution of imaging, and the like.

The smart device 300 additionally includes a transmitter 306 operatively coupled to the user interface 302 and configured to receive the user inputs and/or controls 304 from the user interface 302. The transmitter 306 is configured to wirelessly transmit the user inputs and/or controls 304 to the probe adapter coupled to the smart device 300.

Moreover, the smart device 300 further includes a wireless receiver 308. In one embodiment, the receiver 308 may receive partially-processed image data from the probe adapter. In another embodiment, the receiver 308 may receive fully processed image data representative of an image of the ROI in the subject from the probe adapter. Furthermore, the partially-processed image data may be the partially-processed image data 224 and the fully-processed image data may be the fully-processed image data 226 of FIG. 2.

The smart device 300 additionally includes a processing subsystem 310 operatively coupled to the receiver 308. In one embodiment, the processing subsystem 310 is configured to receive the partially-processed image data from the receiver 308. Additionally, the processing subsystem 310 is configured to process the partially-processed image data to generate an image of the ROI in the subject. In another embodiment, the processing subsystem 310 is configured to receive the fully-processed image data representative of the image of the ROI in the subject from the receiver 308.

Further, the smart device 300 includes a display device 312 operatively coupled to the processing subsystem 310. The display device 312 is configured to receive the image from the processing subsystem 310, and display the image.

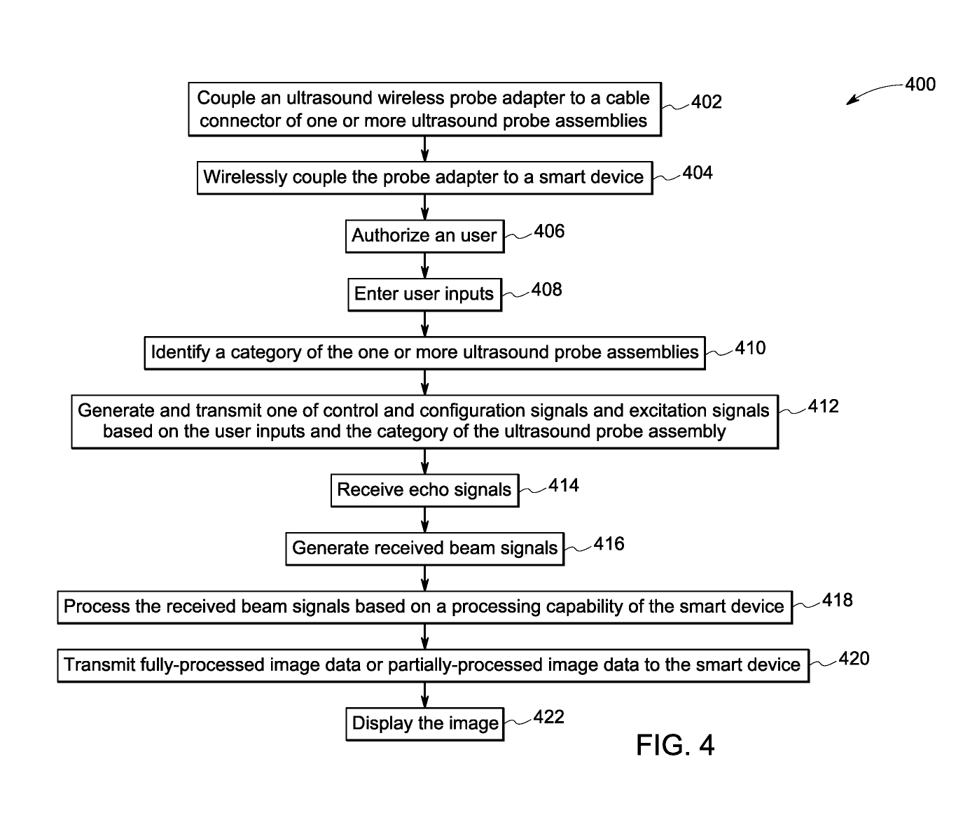

FIG. 4 is a flowchart of a method 400 of imaging using the exemplary wireless ultrasound probe adapter 106 (see FIG. 1), in accordance with aspects of the present specification. The method 400 of FIG. 4 may be described with reference to the components of FIGS. 1-3.

As previously noted with reference to FIG. 1, the wireless probe adapter 106 includes the first coupling unit 116, the microcontroller 118, and the second coupling unit 130. At block 402, an ultrasound probe adapter such as the wireless probe adapter 106 may be coupled to the cable connector 114 of the ultrasound probe assembly 104. Particularly, the first coupling unit 116 of the probe adapter 106 may be detachably coupled to the cable connector 114 of the ultrasound probe assembly 104. As previously noted, the probe adapter 106 is designed to be detachably couplable to one or more categories of ultrasound probe assemblies.

Subsequently, at block 404, the probe adapter 106 may be wirelessly coupled to a smart device. The smart device, for example may be the smart device 108, 300. The probe adapter 106, for example, may be wirelessly coupled to the smart device 108 via the second coupling unit 130 of the probe adapter 106. Furthermore, in accordance with aspects of the present specification, it may be desirable to authenticate and/or authorize a user of the probe adapter, as indicated by block 406. In one embodiment, the probe adapter 106 may be configured to authenticate credentials of the user via a password, biometrics, and the like. Once the credentials of the user are authenticated, the user may be allowed to use the wireless probe adapter 106.

At block 408, inputs may be entered by a user. As previously noted, the user inputs may be used to control operation of the probe adapter 106 and/or the probe assembly 104.

Subsequently, at block 410, a category of the ultrasound probe assembly 104 may be identified. The category of the ultrasound probe assembly 104, for example, may be determined based on a presence or absence of active TX/RX electronics in the ultrasound probe assembly 104. Although in the example of FIG. 4, block 410 is shown as a separate step/block, it may be noted that in certain embodiments, block 410 may be automatically performed subsequent to the coupling of the probe adapter 106 to the ultrasound probe assembly 104.

Moreover, at block 412, the probe adapter 106 may generate and transmit control and configuration signals 121 or excitation signals 120 based on the user inputs (see block 408) and the identified category of the ultrasound probe assembly 104 (see block 410). For example, if the category of the ultrasound probe assembly 104 is identified as including the active TX/RX electronics, then the probe adapter 106 generates the control and configuration signals 121. Similarly, when the category of the ultrasound probe assembly 104 is identified as not including the transmit/receive (TX/RX) electronics, the probe adapter 106 generates the excitation signals 120.

Further, the wireless coupling of the probe adapter 106 to the smart device 108 and authorization of the user enables the probe adapter 106 to transmit the control and configuration signals 121 or the excitation signals 120 to the ultrasound probe assembly 104 to initiate emission of the acoustic signals 122 towards a region of interest in the subject 102. The emission of the acoustic signals 122 results in generation of echo signals 124, 213. At block 414, the echo signals 124, 213 may be received by the probe adapter 106 from the ultrasound probe assembly 104. Subsequently, at block 416, received beam signals 215 may be generated based on the received echo signals 213. The received beam signals 215, for example, may be generated by the receive beamforming unit 220 of the probe adapter 200.

Subsequently, at block 418, the received beam signals 215 may be processed by the probe adapter 106 based on a processing capability of the smart device 108. In accordance with aspects of the present specification, the probe adapter 106 is configured to partially process or fully process the received beam signals 215 to respectively generate partially-processed image data 224 or fully-processed data 226 based on the processing capability of the smart device 108. More particularly, if the processing capability of the smart device 108 is substantially faster than the processing capability of the probe adapter 106, then the probe adapter 106 is configured to partially process the received beam signals 215 to generate the partially-processed image data 224. Alternatively, if the processing capability of the smart device 108 is either lower than the processing capability of the image processor 222 or the smart device 108 is incapable of processing the received beam signals 215, then the probe adapter 106 is configured to fully process the received beam signals 215 to generate the fully-processed image data 226. As previously noted, the fully-processed image data 226 is representative of the image.

Subsequently, at block 420, the fully-processed image data 226 or the partially-processed image data 224 may be transmitted to the smart device 108. In one embodiment, the smart device 108 may further process the partially-processed image data 224 to generate the image (not shown) for display. In another embodiment, the smart device 108 may generate the image based on the fully-processed image data 226. In addition, at block 422, the image of may be visualized on a display device of the smart device 108. The image may be used by a clinician to evaluate a condition of the subject, provide a diagnosis, and/or track progression of a disease state in the subject. In certain embodiments, the image may be communicated to a clinician at a remote location.

In accordance with further aspects of the present specification, a kit for imaging is presented. Such a kit may include an ultrasound wireless probe adapter, such as the exemplary ultrasound wireless probe adapter 106 of FIG. 1. As will be appreciated, currently, there exists a huge installed base of existing conventional wired/tethered probes. The kit including the probe adapter 106 may be employed to provide a cost-effective solution to upgrade the huge installed base of existing conventional probes to a wireless (untethered) and compact ultrasound probe/probe assembly. In particular, the probe adapter 106 may be retrofit to currently existing tethered ultrasound probes to upgrade these probes to wireless probes in a simple and cost-effective manner.

As previously noted, the probe adapter 106 includes the first coupling unit 116, the microcontroller 118, and the second coupling unit 130. The first coupling unit 116 is configured to aid in detachably coupling the probe adapter to an ultrasound probe assembly, such as the ultrasound probe assembly 104. The microcontroller 118 is operatively coupled to the first coupling unit 116 and configured to transmit control and configuration signals or excitation signals to the ultrasound probe assembly 104 to initiate emission of acoustic signals towards an ROI in a subject. The microcontroller 118 is additionally configured to receive echo signals generated in response to the transmitted control and configuration signals or the excitation signals from the ultrasound probe assemblies and perform one of partial processing and full processing of the echo signals generate one of partially-processed image data or fully-processed image data.

Furthermore, the second coupling unit 130 is operatively coupled to the microcontroller 118 and configured to wirelessly couple the probe adapter 106 to a smart device, such as the smart device 108. The second coupling unit 130 aids the probe adapter 106 in transmitting portions of the partially-processed image data and/or the fully-processed image data to the smart device 108 for generation and/or display of an image of the ROI in the subject.

Furthermore, the foregoing examples, demonstrations, and process steps such as those that may be performed by the system may be implemented by suitable code on a processor-based system, such as a general-purpose or special-purpose computer. It should also be noted that different implementations of the present technique may perform some or all of the steps described herein in different orders or substantially concurrently, that is, in parallel. Furthermore, the functions may be implemented in a variety of programming languages, including but not limited to C++ or Java. Such code may be stored or adapted for storage on one or more tangible, machine readable media, such as on data repository chips, local or remote hard disks, optical disks (that is, CDs or DVDs), memory or other media, which may be accessed by a processor-based system to execute the stored code. Note that the tangible media may comprise paper or another suitable medium upon which the instructions are printed. For instance, the instructions may be electronically captured via optical scanning of the paper or other medium, then compiled, interpreted or otherwise processed in a suitable manner if necessary, and then stored in the data repository or memory.

Various embodiments of a wireless probe adapter are presented. The ultrasound wireless probe adapter is configured to convert a conventional, wired ultrasound probe assembly to a wireless ultrasound probe assembly. Particularly, operatively coupling the ultrasound wireless probe adapter to a wired ultrasound probe assembly enables wireless coupling of the ultrasound wireless probe adapter and the wired ultrasound probe assembly to a smart device. The probe adapter provides a cost-effective solution to upgrade a huge installed base of existing conventional probes to a wireless (untethered), compact, low cost, and easily upgradable ultrasound imaging system.

Additionally, use of the wireless adapter may allow for extended battery life and improved thermal performance compared to digital probes since the system electronics are housed in the wireless adapter (away from the patient) rather than inside the ultrasound probe. The exemplary wireless probe adapter leverages the ubiquitous presence of the smart devices to provide a compact, cost-effective, and easy to transport imaging system. Furthermore, the probe adapter includes intelligence to enable the probe adapter to choose whether the probe adapter needs to partially process received beam signals to generate partially-processed image data or fully process the received beam signals to generate fully-processed image data based on a processing capability of the smart device.

Moreover, the probe adapter is capable of operating with different categories of ultrasound probe assemblies. For example, if an ultrasound probe assembly coupled to the probe adapter does not include transmit/receive electronics, then the probe adapter may perform the functions of the transmit/receive electronics not present in the ultrasound probe assembly. However, if the ultrasound probe assembly coupled to the probe adapter includes transmit/receive electronics, then the probe adapter may bypass performing the functions of the transmit/receive electronics present in the ultrasound probe assembly.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An ultrasound wireless probe adapter, comprising:
a first coupling unit configured to detachably couple the probe adapter to one or more ultrasound probe assemblies;
a second coupling unit configured to wirelessly couple the probe adapter to a smart device;
a microcontroller operatively coupled to the first coupling unit and the second coupling unit and configured to:
wirelessly communicate with the smart device to accept user inputs;
generate and transmit one of excitation signals and control and configuration signals to the one or more ultrasound probe assemblies based on the user inputs and a category of the one or more ultrasound probe assemblies to initiate emission of acoustic signals towards a region of interest in a subject;
receive echo signals generated by the one or more ultrasound probe assemblies in response to one of the transmitted excitation signals or the transmitted control and configuration signals;
process received beam signals based on a processing capability of the smart device to generate one of partially-processed image data and fully-processed image data, wherein the received beam signals are generated based on the received echo signals, and
wherein the probe adapter is configured to wirelessly transmit one of the partially-processed image data and the fully-processed image data to the smart device for generation and display of an image of the region of interest in the subject.

2. The probe adapter of claim 1, wherein the microcontroller is further configured to:
determine the category of the one or more ultrasound probe assemblies based on a configuration or a processing capability of the one or more ultrasound probe assemblies; and
generate and transmit one of the excitation signals and the control and configuration signals to the one or more ultrasound probe assemblies based on the determined category of the one or more ultrasound probe assemblies.

3. The probe adapter of claim 1, wherein the microcontroller is further configured to:
determine the category of the one or more ultrasound probe assemblies based on a presence or an absence of transmit/receive electronics in the one or more ultrasound probe assemblies; and
generate and transmit one of the excitation signals and the control and configuration signals to the one or more ultrasound probe assemblies based on the determined category of the one or more ultrasound probe assemblies.

4. The probe adapter of claim 1, wherein the microcontroller further comprises an image processor configured to fully process or partially process the received beam signals based on the processing capability of the smart device.

5. The probe adapter of claim 1, wherein the microcontroller is further configured to:
compare the processing capability of the smart device to a processing capability of the probe adapter; and
partially process or fully process the received beam signals based on the comparison.

6. The probe adapter of claim 1, wherein the microcontroller further comprises:
a thermal management unit configured to manage a temperature of the probe adapter;
a digital identification unit configured to authorize a user of the probe adapter; and
a power supply unit configured to supply electric power to the probe adapter.

7. The probe adapter of claim 1, wherein the probe adapter is characterized by a portable and compact size.

8. The probe adapter of claim 1, wherein the probe adapter is configured to convert wired probe assemblies to wireless probe assemblies.

9. The probe adapter of claim 1, wherein the first coupling unit comprises a female connector or a male connector.

10. The probe adapter of claim 1, wherein a size of the probe adapter is equal to a size of a cable connector of the one or more ultrasound probe assemblies.

11. An ultrasound imaging system, comprising:
one or more ultrasound probe assemblies;
a smart device;
an ultrasound wireless probe adapter comprising:
a first coupling unit configured to detachably couple the probe adapter to the one or more ultrasound probe assemblies;
a second coupling unit configured to wirelessly couple the probe adapter to the smart device;
a microcontroller operatively coupled to the first coupling unit and the second coupling unit and configured to:
wirelessly communicate with the smart device to accept user inputs;
generate and transmit one of excitation signals and control and configuration signals to the one or more ultrasound probe assemblies based on the user inputs and a category of the one or more ultrasound probe assemblies to initiate emission of acoustic signals towards a region of interest in a subject;
receive echo signals generated by the one or more ultrasound probe assemblies in response to the transmitted excitation signals or the transmitted control and configuration signals; and
process received beam signals based on a processing capability of the smart device to generate one of partially-processed image data and fully-processed image data, wherein the received beam signals are generated based on the received echo signals,
wherein the probe adapter is configured to wirelessly transmit one of the partially-processed image data and the fully-processed image data to the smart device for generation and display of an image of the region of interest in the subject.

12. The ultrasound imaging system of claim 11, wherein the one or more ultrasound probe assemblies comprise different categories of ultrasound probe assemblies.

13. The ultrasound imaging system of claim 11, the microcontroller is further configured to:
determine the category of the one or more ultrasound probe assemblies based on a configuration or a processing capability of the one or more ultrasound probe assemblies; and
generate and transmit one of the excitation signals and the control and configuration signals to the one or more ultrasound probe assemblies based on the determined category of the one or more ultrasound probe assemblies.

14. The ultrasound imaging system of claim 11, wherein the microcontroller is further configured to:
determine the category of the one or more ultrasound probe assemblies based on a presence or absence of transmit/receive electronics in the one or more ultrasound probe assemblies; and
generate and transmit one of the excitation signals and the control and configuration signals based on the determined category.

15. The ultrasound imaging system of claim 11, wherein each of the one or more ultrasound probe assemblies comprises:
a probe;
a cable comprising a first end and a second end, wherein the first end of the cable is operatively coupled to the probe; and
a cable connector operatively coupled to the second end of the cable,
wherein the cable connector is selected based on a type of the first coupling unit.

16. The ultrasound imaging system of claim 11, wherein the microcontroller is configured to select between a partial processing or a full processing of the received beam signals based on a comparison of processing capabilities of the probe adapter and the smart device.

17. The ultrasound imaging system of claim 11, wherein the smart device comprises:
a user interface configured to receive user inputs;
a transmitter operatively coupled to the user interface and configured to transmit the user inputs to the probe adapter;
a receiver configured to receive one of the partially-processed image data and the fully-processed image data from the probe adapter, wherein the fully-processed image comprises image data representative of the image of the region of interest in the subject; and
a display device configured to visualize the image of the region of interest in the subject.

18. The ultrasound imaging system of claim 11, wherein the smart device further comprises a processing subsystem operatively coupled to the receiver and configured to further process the partially-processed image data to generate the image of the region of interest in the subject.

19. The ultrasound imaging system of claim 11, wherein the probe adapter is configured to convert wired probe assemblies to wireless probe assemblies.

20. A method for imaging, comprising:
coupling an ultrasound wireless probe adapter to a cable connector of one or more ultrasound probe assemblies, wherein the probe adapter comprises:
a first coupling unit configured to detachably couple the probe adapter to the one or more ultrasound probe assemblies;
a second coupling unit configured to wirelessly couple the probe adapter to a smart device;
a microcontroller operatively coupled to the first coupling unit and the second coupling unit and configured to:
wirelessly communicate with the smart device to accept user inputs;
generate and transmit one of excitation signals and control and configuration signals to the one or more ultrasound probe assemblies based on the user inputs and a category of the one or more ultrasound probe assemblies to initiate emission of acoustic signals towards a region of interest in a subject;
receive echo signals generated by the one or more ultrasound probe assemblies in response to the transmitted excitation signals or the transmitted control and configuration signals; and
process received beam signals based on a processing capability of the smart device to generate one of partially-processed image data and fully-processed image data, wherein the received beam signals are generated based on the received echo signals,
wirelessly coupling the probe adapter to the smart device via the second coupling unit;
authorizing a user of the probe adapter;
generating an image based on one of the partially-processed image data and the fully-processed image data; and
displaying the image on the smart device.
